(19) United States
(12) Patent Application Publication
(10) Pub. No.: US 2007/0106219 A1
(43) Pub. Date: May 10, 2007

(54) Cleveland round tip (CRT) needle

(76) Inventor: Andreas Grabinsky, Silverdale, WA (US)

Correspondence Address: STETINA BRUNDA GARRED & BRUCKER, 75 ENTERPRISE, SUITE 250, ALISO VIEJO, CA, 92656, US

(21) Appl. No.: 11/263,246

(22) Filed: Oct. 31, 2005

(51) Int. Cl. A61M 5/178

(52) U.S. Cl. 604/164.01

(57) ABSTRACT

A needle assembly is provided for mitigating penetration injury during an injection into a desired area. The assembly comprises a cannula and a stylet. The cannula includes an axially-disposed passageway and a cannula rim disposed at a distal end thereof. The cannula rim defines a non-cutting edge. The stylet is tapered to a stylet tip at a leading end thereof. The stylet is removably positionable within the passageway of the cannula with the stylet tip being longitudinally extendable beyond the cannula rim. The leading end and the cannula rim collectively form a piercing head. The piercing head facilitates penetration of the cannula into the desired area. Subsequent to penetration, retraction of the stylet tip from beyond the cannula rim facilitates delivery of the medication through the passageway and exposes the non-cutting edge of the cannula rim to mitigate further penetration.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The present invention relates generally to medical needle assemblies, and more particularly to an improved needle assembly featuring a cannula and stylet each having a rounded, non-cutting tip that is specifically adapted to facilitate penetration to a target nerve area without causing nerve injuries and/or penetrating into surrounding structure.

Epidural steroid injection (ESI) is a well known non-surgical treatment designed to alleviate pain in the neck, arm, low back, and leg caused by irritation of spinal nerves. Typically, an ESI produces long-lasting relief for a patient by delivering an anesthetic agent to the irritated and inflamed spinal nerve. During the ESI process, a needle is introduced into epidural space near the spinal cord, in a designated area as close to the irritated nerve as possible. Upon reaching the designated area, the steroid medication is delivered through the needle to the inflamed nerve. Thus, the patient may be relieved of symptoms caused by inflammation and pressure on the spinal nerves through a non-surgical process. The overriding goal of ESI is to reduce pain so that patients may resume normal activity, which may include additional physical therapy regimens.

As one may expect, the placement of the needle relative to the epidural space may produce varying results and is critical to the effectiveness of the ESI. In fact, there are generally two types of ESI's, with the principal difference being the location of the needle relative to the epidural space. In order to accurately guide the needle through the skin and into the epidural space, x-ray fluoroscopy is typically utilized. Fluoroscopy thus allows the doctor to visually monitor the movement and placement of the needle, thereby allowing the doctor to deliberately and carefully position the needle. See Epidural Steroidal Injection, Mayfield Clinic & Spine Institute, available at www.mayfieldclinic.com/PE-ESI.htm (last visited Jun. 22, 2005).

The first and more traditional type of ESI is called translaminar epidural injection. This type of injection evolved from spinal anesthesia, in which a thin needle is advanced to close proximity of the spinal cord and medication is injected into the spinal fluid itself. For the translaminar epidural injection, the needle is positioned between the lamina of two vertebrae in the midline of the back. The needles will need to penetrate the skin and ligaments between the vertebrae to reach the epidural space. This placement allows the medication to be delivered into the large epidural space surrounding the spinal cord. Thus, the medication may reach both the right and left sides of the nerve root area at the same time. The spinal cord together with the spinal fluid is separated from the epidural space by a sac, called the dura. For spinal anesthesia the dura needs to be penetrated, while for the epidural injection the dura must not be penetrated. This midline approach avoids the risk of nerve or vascular damage, since there are no nerves or vessels in the ligament between the vertebrae. See Epidural Steroidal Injection, Mayfield Clinic & Spine Institute, available at www.mayfieldclinic.com/PE-ESI.htm (last visited Jun. 22, 2005).

The second type of ESI, transforaminal injection, delivers the medication more directly to the inflamed nerve. For transforaminal injection, the needle is positioned on one side of the vertebrae and the needle will pass through muscle tissue, reaching the neural foramen at the side of the spine where the spinal nerve exits the spinal canal. This procedure has been proven to be more useful because it targets specifically the affected nerve and it allows the doctor to avoid scars or obstructions such as bone grafts, metal rods and screws from previous back surgeries. On the side of the spine and within the muscles are nerves and vessels located, which cannot be seen on fluoroscopy and can therefore easily damaged or penetrated by sharp, cutting needles. See Epidural Steroidal Injection, Mayfield Clinic & Spine Institute, available at www.mayfieldclinic.com/PE-ESI.htm (last visited Jun. 22, 2005).

In performing the transforaminal injection various needles have been utilized, which were originally developed for translaminar epidural or spinal injections. Unfortunately, these prior art needles have certain disadvantageous characteristics that may lead to complications and/or ineffective treatment results. A first disadvantage of prior art needles lays the use of sharp cutting points or edges. Although this feature may allow the needle to easily penetrate and reach the desired area, control of the needle is extremely critical because of their sharp points and edges. Although sharp cutting points and edges may be helpful in facilitating penetration of the needle, they also increase the potential risk of undesired penetration and injury to nerves and surrounding structures. For example, a sharp needle may cause nerve injuries or penetrate into the intestine or blood vessels, which may result in complications during the procedure. The adverse outcomes of this regional anesthesia may include temporary nerve injury, paralysis, or death. However, all of these adverse effects may be avoided by ensuring that the needle does not penetrate or damage surrounding structures during the injection. Further, such adverse effects may also be avoided by properly injecting the medication at the desired area.

Additional changes in needle design were made to remedy the first disadvantage of prior art needles, sharp, cutting points and edges. In fact, some of these needles are commonly known as Whitacre, Sprotte and blunt needles. These were designed to minimize the common risk of headache after spinal anesthesia. These needles were designed for spinal anesthesia, but because the blunt tip reduces the risk of improper penetration of the needle into nerves, intestines, or blood vessels, these needles are sometimes used for transforaminal injections. However, these needles also have a certain disadvantage in their design: the medication is delivered to the desired area via a side hole. The side hole of these prior art needles is often located at a distance from the tip of the needle. The delivery mechanism (the side hole) may be imprecise in delivering medication to the desired area, which can be problematic and ineffective for small areas. Thus, although the tip of the needle may reach the target area without penetrating other structures, the delivery of medication may be ineffectual because the side hole may not be precisely positioned within the desired area. In such a case, the medication may not be delivered to the nerve root within the desired area. Another disadvantage associated with these needles is that the tip may be too blunt, making penetration to the desired area more difficult.

Therefore, there is a need in the art for a needle without sharp cutting edges and points, but yet sharp enough to facilitate penetration to the desired area. Additionally, there is a need in the art for a needle that is specifically configured to effectively deliver of medication to the desired area. Finally, there is also a need in the art for a needle assembly utilizing a cannula and stylet wherein the cannula is non-cutting with or without the stylet being inserted therein.

### BRIEF SUMMARY

In accordance with an embodiment of the present invention, a needle assembly is provided for mitigating penetration injury during an injection into a desired area. Such injection may be in regard to a peripheral nerve block, sympathetic nerve block, or a transforaminal injection, or other types of injections. The assembly comprises a cannula and a stylet. The cannula includes an axially-disposed passageway and a cannula rim disposed at a distal end thereof. The cannula rim defines a non-cutting edge. The stylet is tapered to a stylet tip at a leading end thereof. The stylet is removably positionable within the passageway of the cannula with the stylet tip being longitudinally extendable beyond the cannula rim. The leading end and the cannula rim collectively form a piercing head. In use, the. piercing head facilitates penetration of the cannula into the desired area. Subsequent to penetration into the desired area, the stylet tip may be retracted from beyond the cannula rim. This retraction facilitates delivery of the medication through the passageway and exposes the non-cutting edge of the cannula rim. Thus, the exposed non-cutting cannula rim may mitigate further penetration of the needle assembly.

According to an aspect of the present invention, the leading end of the stylet may define an axially convex surface tapering until converging to form the stylet tip. The leading end of the stylet may be formed substantially as a geometric lemon shape. In addition, the stylet tip may be axially aligned with the cannula. The cannula rim may be rounded. Additionally, the cannula rim may be configured as a rounded bevel.

In accordance with another embodiment of the present invention, a needle assembly is provided for mitigating penetration injury during an injection of medication into a desired area. The assembly comprises a cannula and a stylet. The cannula includes an axially-disposed passageway and defines a cannula diameter, a cannula collar, and a distal edge. The cannula diameter decreases from the cannula collar until reaching the distal edge to form a cannula rim. The cannula rim defines a non-cutting edge. The stylet defines a leading end, a stylet collar, and a stylet tip. The stylet tapers to the stylet tip at the leading end. The stylet is removably positionable within the passageway with the stylet collar being approximately adjacent the cannula rim to facilitate penetration of the cannula into the desired area. In use, the stylet tip may be retracted from beyond the cannula rim subsequent to penetration into the desired area to facilitate delivery of the medication through the passageway and to expose the non-cutting edge of the cannula rim to mitigate further penetration.

The leading end of the stylet may define an axially convex surface which tapers until converging to form the stylet tip. Additionally, the stylet may further define a stylet diameter, and the stylet diameter may increasingly decrease from the stylet collar until reaching the stylet tip. The stylet tip may be axially aligned with the cannula. The stylet may define a distal surface having a continuous curvature therealong. Further, the stylet may include a substantially cylindrical stylet body. The cannula diameter may increasingly decrease from the cannula collar until reaching the distal edge. Finally, the cannula rim may further define a rounded inner edge.

In accordance with yet another embodiment of the present invention, a needle assembly is provided for injection of medication into a desired area. The assembly comprises a cannula, a stylet, and a hub. The cannula defines distal and proximal ends and includes an axially-disposed passageway and a cannula rim disposed at the distal end. The cannula rim defines a non-cutting edge. The stylet is tapered to a stylet tip at the leading end thereof. The stylet is positionable within the passageway with the stylet tip being longitudinally extendable beyond the cannula rim. The leading end and the cannula rim collectively forming a piercing head. The hub is attachable to the proximal end of the cannula and includes a bore. The stylet is insertable into the passageway through the bore, and the hub is operative to longitudinally secure the stylet within the passageway upon formation of the piercing head. In use, the piercing head facilitates penetration of the cannula into the desired area. Subsequent to penetration into the desired area, the stylet tip may be retracted from beyond the cannula rim. This retraction facilitates delivery of the medication through the passageway and exposes the non-cutting edge of the cannula rim to mitigate further penetration.

According to an aspect of the present invention, the leading end of the stylet may define an axially convex surface tapering until converging to form the stylet tip. Additionally, the bore may be axially aligned with the cannula. Further, the hub may include a fastener to secure the proximal end of the stylet to the hub upon formation of the piercing head. In this regard, the fastener may be a luer lock. Finally, the hub may further include an indicator. The indicator may be in communication with the stylet tip and may be operative to visually indicate position of the stylet tip in relation to the cannula.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 a view of a needle assembly for mitigating penetration injury during an injection in accordance with an aspect of the present invention;

FIG. 2 shows a distal end of the assembly including a stylet and a cannula in accordance with another aspect of the present invention;

FIG. 3 is a cross sectional view of the distal end of the assembly showing a tip of the stylet being longitudinally disposed beyond rim of the cannula to facilitate penetration of the assembly into the desired area in accordance with another aspect of the present invention;

FIG. 4 is a cross sectional view of the distal end of the assembly showing the stylet tip being longitudinally withdrawn from beyond the cannula rim to facilitate passage of medication and to mitigate further penetration of the assembly in accordance with another aspect of the present invention;

FIG. 5 is a cross sectional view of a section of the assembly showing an enlarged view of the cannula rim in accordance with another aspect of the present invention; and

FIG. 6 is a cross sectional view of the distal end of the assembly wherein the cannula rim is configured as a rounded bevel.

### DETAILED DESCRIPTION

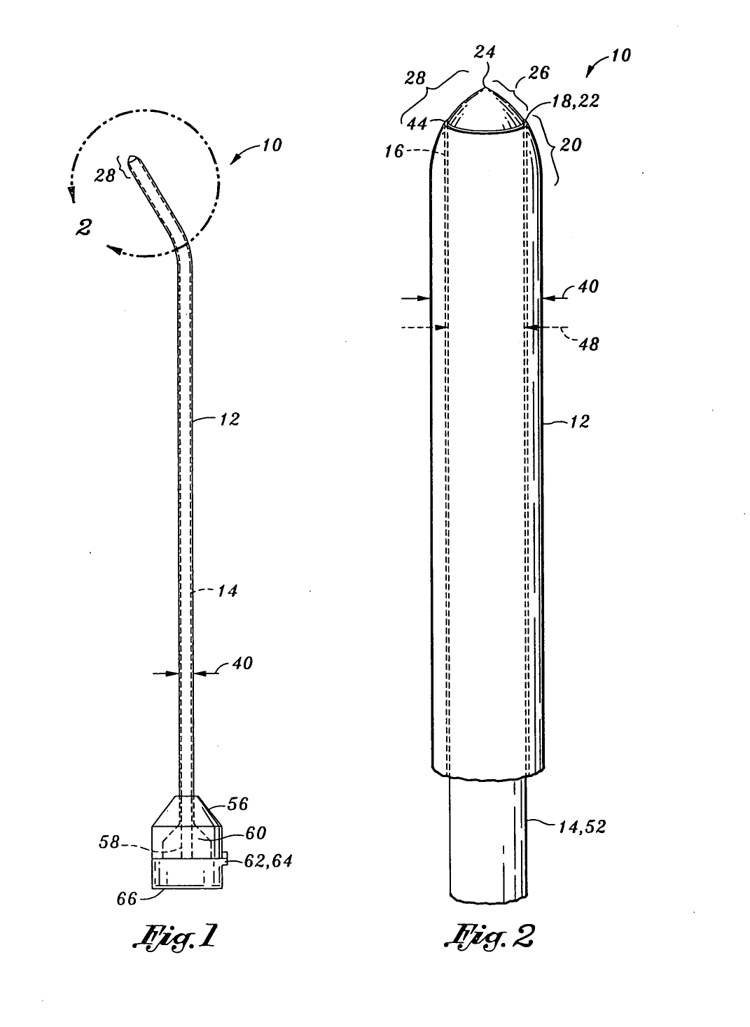

Referring now to the drawings wherein the showings are for purposes of illustrating the preferred embodiment invention only and not for purposes of limiting the same, FIG. 1 illustrates a needle assembly 10 for mitigating penetration injury during an injection into a desired area. The embodiments of the present invention described herein may easily penetrate to the desired area, such as a target nerve area adjacent a spinal cord, as may be common in peripheral nerve blocks, sympathetic nerve blocks, and transforaminal injections. As mentioned above, such injections may include those for peripheral nerve blocks, sympathetic nerve blocks, or transforaminal injections, or other types of injections. However, in contrast to prior art needles, embodiment for the present invention are configured to avoid further penetration into the desired area subsequent to the assembly 10 being properly positioned within the desired area, thereby substantially eliminating unintentional penetration and consequent penetration injuries resulting therefrom. In addition, embodiments of the present invention are also believed to be superior to various prior art needle assemblies due to the accuracy of the delivery of medication, which is delivered at a tip of the needle assembly 10, as opposed to from a side hole of prior art needles. Thus, embodiments of the present invention provide at least these two specific novel and advantageous aspects over previous needle assemblies.

The needle assembly 10 of embodiments of the present invention may be utilized beneficially for all nerve blocks in which peripheral nerves or vessels are potentially in the path of the needle assembly 10 and therefore could be damaged or injected into. Although the needle assembly 10 may be utilized in spinal anesthesia or interlaminar epidural injection, these applications may not benefit as substantially because in such applications, there are basically no vessels or nerves in the path of the needle assembly 10. However, the needle assembly 10 may be very useful in applications of peripheral nerve blocks, sympathetic nerve blocks, and transforaminal injections. Although the length of the needle assembly 10 may be altered depending on the given application, such is within the scope of the present invention. In peripheral nerve blocks, sympathetic nerve blocks, and transforaminal injections, the tip of the needle assembly 10 is typically advanced through muscle tissue in which nerves and arteries may be embedded, and the target area for such injections is a nerve. Thus, a skilled doctor must position the tip of the needle assembly 10 as close as possible to the nerve without damaging the target area/nerve. As will be appreciated by one of skill in the art, implementations of the present invention are extremely beneficial and offer a higher degree of safety for all peripheral nerve blocks, transforaminal/selective nerve blocks and sympathetic nerve blocks.

Referring to FIGS. 2-3, the needle assembly 10 comprises a cannula 12 and a stylet 14. The assembly 10 may be linear, bent (as shown in FIG. 1), or otherwise shaped to facilitate the use thereof. The cannula 12 includes an axially-disposed passageway 16 and a cannula rim 18 being disposed at a distal end 20 of the cannula 12. The cannula rim 18 defines a non-cutting edge 22, which ensures that the cannula 12, when used without the stylet 14, will not penetrate further after being positioned into the desired area. The stylet 14 tapers to a stylet tip 24 at a leading end 26 thereof. The stylet tip 14 is preferably not pointed; instead, the stylet tip 14 may be rounded. In this regard, the stylet tip 14 may be rounded and not pointed in order to not directly puncture blood vessels and other fibrous elements and to enable a tactile feedback of being pushed against these structures. The stylet 14 is removably positionable within the passageway 16 of the cannula 12 with the stylet tip 24 being longitudinally extendable beyond the cannula rim 18. In this regard, it is contemplated that the stylet 14 may radially occupy a sufficient volume of the passageway 16 in order to prevent the entry of fluids or other materials thereinto during penetration of the assembly 10 into a patient. The leading end 26 of the stylet 14 and the cannula rim 18 collectively form a piercing head 28.

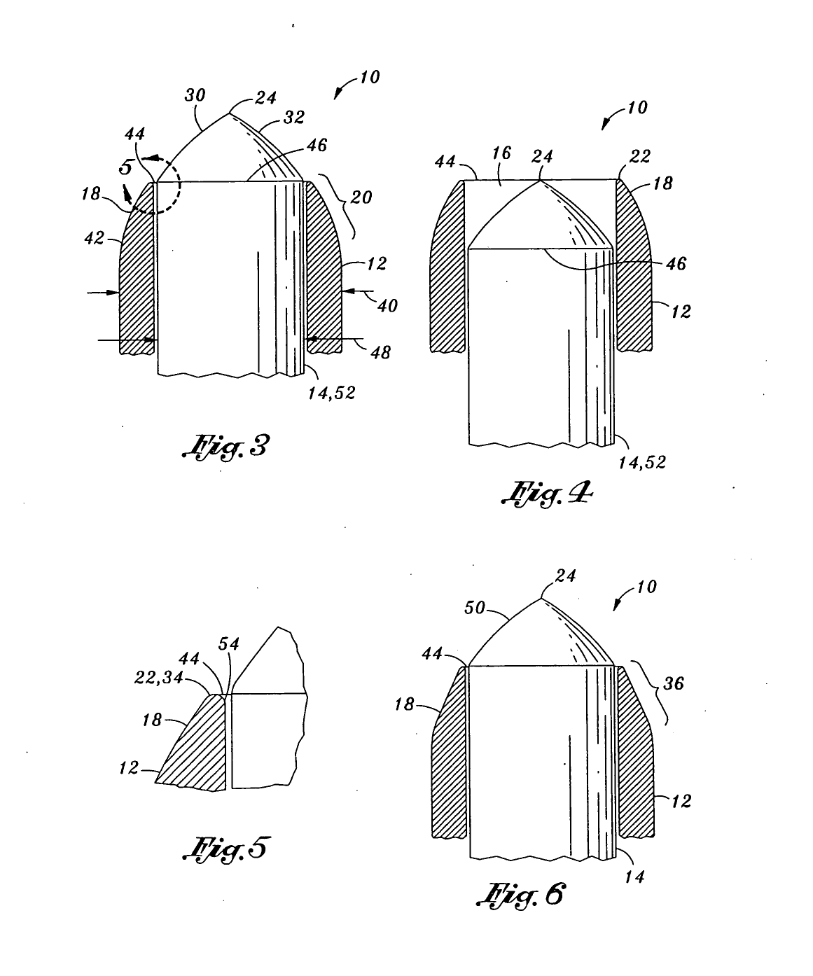

In use, the piercing head 28 facilitates penetration of the cannula 12 into the desired area. Subsequent to the piercing head 28 reaching the desired area, retraction of the stylet tip 24 from beyond the cannula rim 18 facilitates delivery of the medication through the passageway 16, as shown in FIG. 4. Additionally, such retraction serves to disassemble the piercing head 28, which may prevent further penetration of the assembly 10 into the desired area or other regions, nerves, and/or other tissue, as discussed below. After retraction, medication may thus flow through the passageway 16 out from the distal end 20 of the cannula 12 and into the desired area.

The piercing head 28 may also be utilized to locate nerves by sending a small dose of electrical current (0.1 to 0.5 mAmp) through an electrode. The electrode may be disposed at the stylet tip 24 and/or the cannula rim 18, for example. In this regard, there may be a single electrode or several. In use, the stylet 14 and/or cannula 12 would preferably be insulated along the entire length thereof, except for the stylet tip 24 and/or the cannula rim 18, as necessary. The electrical current may be provided to the electrode as through an electrical wire disposed within the stylet 14 and/or cannula 12. The electrical wire is preferably in electrical communication with a power source and a current regulator for selectively regulating the electrical current delivered at the electrode. Thus, the stylet tip 24 and/or cannula rim 18 (collectively the piercing head 28) may also be utilized to locate nerves. Other modifications may be performed and implemented.

It is contemplated that the needle assembly 10 may be configured to allow delivery of medication through the cannula 12 either upon complete removal of the stylet 14 therefrom, or upon partial removal therefrom. For example, in some implementations, the stylet tip 24 may be partially withdrawn from beyond the cannula rim 18, whereupon the stylet 14 may be rotated in order to allow medication to pass through a grooved portion or other aspect of the stylet 14 and the cannula 12. Indeed, one of skill in the art may develop several configurations for facilitating passage of the medication to the desired area through the distal end 20 of the cannula 12, such as by varying the shape and configuration of the cannula 12 and stylet 14 to facilitate passage of medication upon the stylet reaching a certain position within the cannula 12, or other various modifications. As mentioned previously, the direct delivery of medication accomplished hereby is believed to improve the accuracy and the overall effectiveness of the injection, whether it is a peripheral nerve block, sympathetic nerve block, and transforaminal injection.

In addition, as also shown in FIG. 4, retraction of the stylet tip 24 from beyond the cannula rim 18 exposes the non-cutting edge 22 of the cannula rim 18, which mitigates further penetration of the assembly 10 into the desired area or other regions, nerves, and/or other tissue during the injection. These advantageous aspects of the present invention, as well as other aspects described herein, provide superior qualities and characteristics over previous needle assemblies and fill various needs in the art.

As illustrated in FIGS. 2-3, it is contemplated that the piercing head 28 of the needle assembly 10 may be collectively defined by the leading end 26 of the stylet 14 and the cannula rim 18. In this regard, the leading end 26 of the stylet 14 may define an axially convex surface 30 tapering until converging to form the stylet tip 24. Thus, the curvature of the surface may be variously configured, and it is contemplated that various shapes and geometries may be implemented in order to achieve varying results. In particular, it is preferred that the curvature of the leading end 26 of the stylet 14 be substantially the same as the curvature of the distal end 20 of the cannula 12, as illustrated in FIG. 2. Thus, although not shown, it may be visualized that both the leading end 26 and the distal end 20 may share a common radius with the same center. In this regard, a person of skill in the art may utilize the teachings herein to develop shapes that are considered to be optimal for certain uses. Thus, although it is contemplated that the leading end 26 of the stylet 14 may define a conical surface that tapers until converging to form the stylet tip 24, according to a preferred embodiment of the present invention, the leading end 26 of the stylet 14 defines the axially convex surface 30. In addition, it is contemplated that the leading end 26 of the stylet 14 may be formed substantially as a geometric lemon shape 32. Although FIGS. 2-3 show a smooth axially convex surface 30, it is also contemplated that the surface may be textured, such as to incorporate grooves, circumferential ribs, or other modification to enhance the penetration ability or other quality of the needle assembly 10. Furthermore, it is contemplated that the leading end 26 of the stylet 14 may also be modified depending on the shape and configuration of the cannula rim 18. Thus, the leading end 26 and the cannula rim 18 may be variously configured to incorporate other advantageous implementations of the present invention.

In accordance with an aspect of the present invention, the stylet tip 24 may be axially aligned with the cannula 12, as illustrated in FIG. 2. Nevertheless, it is also contemplated that the stylet tip 24 may also be aligned off-axis with respect to the cannula 12. Thus, the leading end 26 of the stylet 14 may be variously modified, as indicated above, by one of skill in the art, to incorporate the teachings of implementations of the present invention.

Referring now to FIG. 5, it is contemplated that the cannula rim 18 may be rounded 34. As indicated above, the cannula rim 18 defines the non-cutting edge 22 which is configured to prevent further penetration of the assembly 10 while being positioned in the desired area. Thus, although a blunt edge is a contemplated alternative to being rounded 34, as mentioned above, implementations of the present invention seek to achieve a configuration intermediate a blunt edge and a pointed edge. With this principle in mind, the cannula rim 18 may be variously configured in order to form the piercing head 28. The piercing head 28 should be operative to easily penetrate toward and into the desired area without being as pointed or sharp as alternative needle assemblies known in the art, which often cause penetration injuries. Because the preferred application of embodiments of the present invention is for use in injections such as peripheral nerve blocks, sympathetic nerve blocks, and transforaminal injections, the piercing head 28 need not be as sharp or pointed as other needle assemblies which are utilized to pierce bone or quickly and easily cut into the patient. As illustrated in FIG. 6, the cannula rim 18 may be configured as a rounded bevel 36. Indeed, as shown in the side view of the cannula rim 18 in FIGS. 3-6, it is contemplated that other shapes and curves may be utilized to define the cannula rim 18, according to user requirements and other considerations.

In accordance with another embodiment of the present invention, a needle assembly 10 is provided for mitigating penetration on an injury during an injection of medication into a desired area. Such injection may be a peripheral nerve block, sympathetic nerve block, or transforaminal injection. The assembly 10 comprises a cannula 12 and a stylet 14. The assembly 10 may be linear, bent (as shown in FIG. 1), or otherwise shaped to facilitate the use thereof. The cannula 12 includes an axially-disposed passageway 16 and defines a cannula diameter 40, a cannula collar 42, and a distal edge 44. It is contemplated that the cannula 12 may be cylindrical and define a substantially constant diameter along its length as it approaches the distal edge 44, as shown in FIGS. 1 and 2. The cannula diameter 40 may then decrease from the cannula collar 42 until reaching the distal edge 44 to form a cannula rim 18. The cannula collar 42 may be defined as the location along the cannula 12 at which the cannula diameter 40 substantially begins to taper or decrease approaching the distal edge 44. Although it is contemplated that the shape along the cannula 12 may vary approaching the distal edge 44, in a preferred embodiment, the cannula diameter 40 may follow a substantially smooth line or curve, as shown in FIGS. 2-4 and 6. In this regard, the cannula 12 may be configured to be blunt without the stylet 14 inserted therein. In particular, due to this configuration, the cannula rim 18 thus defines a non-cutting edge 22.

As shown in FIG. 3, the stylet 14 defines a leading end 26, a stylet collar 46, and a stylet tip 24. The stylet 14 tapers to the stylet tip 24 at the leading end 26. In use, the stylet 14 is removably positionable within the passageway 16. As similarly mentioned above with regard to the cannula 12, it is contemplated that the stylet 14 may be cylindrical and define a substantially constant stylet diameter 48 along its length as it approaches the leading end 26, as shown in FIGS. 2-4. The stylet collar 46 may be defined as the location along the stylet 14 at which the stylet diameter 48 substantially begins to taper or decrease approaching the leading end 26.

Prior to insertion of the assembly 10 into the patient, the stylet collar 46 should be-positioned approximately adjacent to the cannula rim 18, as shown in FIGS. 2-3, in order to facilitate penetration of the assembly 10 into the desired area. Subsequent to penetration into the desired area, the stylet tip 24 may be retracted from beyond the cannula rim 18, as illustrated in FIG. 4, in order to facilitate delivery of the medication through the passageway 16. As also shown in FIG. 4, retraction of the stylet tip 24 also exposes the non-cutting edge 22 of the cannula rim 18, thereby mitigating further penetration of the assembly 10 into the desired area. As mentioned previously, several modifications to the stylet 14 and cannula 12 may be performed in order to facilitate delivery of the medication. Additionally, after the assembly 10 has been properly positioned in the desired area of a patient and the stylet 14 has been removed from the cannula 12, the non-cutting edge 22 and configuration of the cannula 12 may mitigate against penetration injuries.

In accordance with an implementation of the present invention, as shown in FIG. 3, the leading end 26 of the stylet 14 may define an axially convex surface 30 which tapers until converging to form the stylet tip 24. Additionally, the stylet 14 may define a stylet diameter 48. As shown in FIGS. 3-4, the stylet diameter 48 may increasingly decrease from the stylet collar 46 until reaching the stylet tip 24. In this manner, it is contemplated that the stylet 14 may facilitate penetration of the assembly 10 without the assembly 10 being too sharp or pointed, as in the prior art, which often results in penetration injuries.

In accordance to yet another aspect of the present invention, the stylet tip 24 may be axially aligned with respect to the cannula 12. However, it is also contemplated that the stylet tip 24 may be aligned off-axis with respect to the cannula 12. Thus, it is contemplated that the geometry of the leading end 26 of the stylet 14 may be variously modified by one of skill in the art in order to achieve results within the scope of embodiments of the present invention. Further, as shown in FIG. 6, the stylet 14 may define a distal surface 50 having a continuous curvature therealong. This particular feature, as similarly mentioned above, may facilitate penetration of the assembly 10 into the desired area. In addition, such configuration may also facilitate insertion of the stylet 14 into the cannula 12 during manufacturing. Further, the stylet 14 may include a substantially cylindrical stylet body 52, as shown in FIGS. 2-4.

In accordance with yet another aspect of the present invention, the cannula diameter 40 may increasingly decrease from the cannula collar 42 until reaching the distal edge 44. The rate of decrease of the cannula diameter 40 may be variously configured in order to facilitate penetration of the assembly 10 as well as to prevent further penetration of the assembly 10 after retraction of the stylet tip 24 from beyond the cannula rim 18 subsequent to penetration into the desired area. In addition, the cannula rim 18 may further define a rounded inner edge 54, which may form an aspect of the non-cutting edge 22 useful to mitigate against further penetration of the assembly 10 upon insertion into the desired area.

In accordance with yet another embodiment of the present invention, a needle assembly 10 is provided for injection of medication into a desired area. As shown in FIG. 1, the assembly 10 comprises a cannula 12, a stylet 14, and a hub 56. The cannula 12 defines distal and proximal ends 20, 58 and includes an axially-disposed passageway 16 and a cannula rim 18. The cannula rim 18 is disposed at the distal end 20 of the cannula 12 and defines a non-cutting edge 22. The stylet 14 is tapered to a stylet tip 24 at a leading end 26 thereof. In this regard, the leading end 26 of the stylet 14 may define an axially convex surface 30 tapering until converging to form the stylet tip 24. The stylet 14 is positionable within the passageway 16 with the stylet tip 24 being longitudinally extendable beyond the cannula rim 18. The leading end 26 and the cannula rim 18 collectively form a piercing head 28. The hub 56 is attachable to the proximal end 58 of the cannula 12 and includes a bore 60. The stylet 14 is insertable into the passageway 16 through the bore 60. In addition, the hub 56 is operative to longitudinally secure the stylet 14 within the passageway 16 upon formation of the piercing head 28.

In use, the piercing head 28 facilitates penetration of the assembly 10 into the desired area. Subsequent to penetration into the desired area, as shown in FIG. 4, retraction of the stylet tip 24 from beyond the cannula rim 18 facilitates delivery of medication through the passageway 16. In addition, as also shown in FIG. 4, retraction of the stylet tip 24 also exposes the non-cutting edge 22 of the cannula rim 18 to mitigate further penetration.

As illustrated in FIG. 1, the bore 60 may be axially aligned with the cannula 12. Such feature may facilitate insertion and retraction of the stylet 14 from the cannula 12. In addition, the hub 56 may further include a fastener 62 to secure the proximal end 58 of the stylet 14 to the hub 56 upon formation of the piercing head 28. Although it is contemplated that the proximal end 58 of the stylet 14 may be secured to the hub 56 utilizing a variety of methods and devices known in the art, it is contemplated that in a preferred embodiment of the present invention, the fastener 62 may be a luer lock 64.

In accordance with yet another aspect of the present invention, the assembly 10 may be configured to indicate when the assembly 10 has reached the desired area. In one embodiment, the hub 56 may further include an indicator 66 for this purpose. For example, the indicator 66 may be in communication with the stylet tip 24 and may be operative to visually indicate position of the stylet tip 24 in relation to a portion of the cannula 12, such as the cannula rim 18. Thus, the indicator 66 may act in response to movement of the stylet tip 24 with respect to the cannula 12, such as when the stylet tip 24 touches a given area.

It is contemplated that the indicator 66 may be variously configured and modified according to one of skill in the art. The above description is given by way of example, and not limitation. Given the above disclosure, one skilled in the art could devise variations that are within the scope of the invention disclosed herein, including various ways of performing injections such as peripheral nerve block, sympathetic nerve block, and transforaminal injections, or other uses of implementations of the present invention in surgical procedures. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A needle assembly for mitigating penetration injury during an injection into a desired area, the assembly comprising:
a cannula including an axially-disposed passageway and a cannula rim being disposed at a distal end thereof, the cannula rim defining a non-cutting edge; and
a stylet being tapered to a stylet tip at a leading end thereof, the stylet being removably positionable within the passageway of the cannula with the stylet tip being longitudinally extendable beyond the cannula rim, the leading end and the cannula rim collectively forming a piercing head,
wherein the piercing head facilitates penetration of the cannula into the desired area, retraction of the stylet tip from beyond the cannula rim subsequent to penetration into the desired area facilitating delivery of the medication through the passageway and exposing the non-cutting edge of the cannula rim to mitigate further penetration.

2. The assembly of claim 1 wherein the leading end of the stylet defines an axially convex surface tapering until converging to form the stylet tip.

3. The assembly of claim 1 wherein the leading end of the stylet is formed substantially as a geometric lemon shape.

4. The assembly of claim 1 wherein the stylet tip is axially aligned with the cannula.

5. The assembly of claim 1 wherein the cannula rim is rounded.

6. The assembly of claim 1 wherein the cannula rim is configured as a rounded bevel.

7. A needle assembly for mitigating penetration injury during an injection of medication into a desired area, the assembly comprising:
a cannula including an axially-disposed passageway and defining a cannula diameter, a cannula collar, and a distal edge, the cannula diameter decreasing from the cannula collar until reaching the distal edge to form a cannula rim, the cannula rim defining a non-cutting edge; and
a stylet defining a leading end, a stylet collar, and a stylet tip, the stylet tapering to the stylet tip at the leading end,
wherein the stylet is removably positionable within the passageway with the stylet collar being approximately adjacent the cannula rim to facilitate penetration of the cannula into the desired area, retraction of the stylet tip from beyond the cannula rim subsequent to penetration into the desired area facilitating delivery of the medication through the passageway and exposing the non-cutting edge of the cannula rim to mitigate further penetration.

8. The assembly of claim 7 wherein the leading end of the stylet defines an axially convex surface which tapers until converging to form the stylet tip.

9. The assembly of claim 7 wherein the stylet further defines a stylet diameter, the stylet diameter increasingly decreasing from the stylet collar until reaching the stylet tip.

10. The assembly of claim 7 wherein the stylet tip is axially aligned with the cannula.

11. The assembly of claim 7 wherein the stylet defines a distal surface having a continuous curvature therealong.

12. The assembly of claim 7 wherein the stylet includes a substantially cylindrical stylet body.

13. The assembly of claim 7 wherein the cannula diameter increasingly decreases from the cannula collar until reaching the distal edge.

14. The assembly of claim 7 wherein the cannula rim further defines a rounded inner edge.

15. A needle assembly for injection of medication into a desired area, the assembly comprising:
a cannula defining distal and proximal ends and including an axially-disposed passageway and a cannula rim being disposed at the distal end, the cannula rim defining a non-cutting edge;
a stylet being tapered to a stylet tip at the leading end thereof, the stylet being positionable within the passageway with the stylet tip being longitudinally extendable beyond the cannula rim, the leading end and the cannula rim collectively forming a piercing head; and
a hub being attachable to the proximal end of the cannula and including a bore, the stylet being insertable into the passageway through the bore, the hub being operative to longitudinally secure the stylet within the passageway upon formation of the piercing head,
wherein the piercing head facilitates penetration of the cannula into the desired area, retraction of the stylet tip from beyond the cannula rim subsequent to penetration into the desired area facilitating delivery of the medication through the passageway and exposing the non-cutting edge of the cannula rim to mitigate further penetration.

16. The assembly of claim 15 wherein the leading end of the stylet defines an axially convex surface tapering until converging to form the stylet tip.

17. The assembly of claim 15 wherein the bore is axially aligned with the cannula.

18. The assembly of claim 15 wherein the hub further includes a fastener to secure the proximal end of the stylet to the hub upon formation of the piercing head.

19. The assembly of claim 15 wherein the fastener is a luer lock.

20. The assembly of claim 15 wherein the hub further includes an indicator, the indicator being in communication with the stylet tip and being operative to visually indicate position of the stylet tip in relation to the cannula.
